(19) United States
(12) Patent Application Publication
(10) Pub. No.: US 2008/0096832 A1
(43) Pub. Date: Apr. 24, 2008

(54) Method for Improving the Pharmacokinetics of an NNRTI

(75) Inventor: Michael Graham Cordingley, Rosemere (CA)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International GmbH, Ingelheim (DE)

(21) Appl. No.: 11/923,699

(22) Filed: Oct. 25, 2007

Related U.S. Application Data
(63) Continuation of application No. 10/736,301, filed on Dec. 15, 2003, now abandoned.
(60) Provisional application No. 60/433,690, filed on Dec. 16, 2002.

(51) Int. Cl. A61K 31/7048; A61K 31/551; A61P 31/18; A61K 31/554

(52) U.S. Cl. 514/29; 514/211.07; 514/220

(57) ABSTRACT

An improved method for using a NNRTI in the treatment of HIV-1 infection, comprising administering to a human, needing treatment for HIV-1 infection, a therapeutically effective amount of said NNRTI or a pharmaceutically acceptable salt thereof, and an amount of an inhibitor of the cytochromes P450 that is sufficient to elevate, enhance, or extend plasma concentrations of said NNRTI.

### CROSS-REFERENCE TO RELATED APPLICATIONS

This application is a continuation of U.S. Ser. No. 10/736,301, filed on Dec. 15, 2003 which claims the benefit of U.S. Provisional Application Ser. No. 60/433,690 filed on Dec. 16, 2002.

### FIELD OF THE INVENTION

The present invention relates to an improved method for using the compound of the formula I in the treatment of HIV-1 infection.

### BACKGROUND OF THE INVENTION

The compound of the formula I is a non-nucleoside HIV-1 reverse transcriptase inhibitor. Its chemical name is 5,11-Dihydro-11-ethyl-5-methyl-8- {2-{(1-oxido-4-quinolinyl)oxy}ethyl}-6H-dipyrido[3,2-b:2′,3′-e] [1,4]diazepin-6-one and its chemical structure is as depicted below.

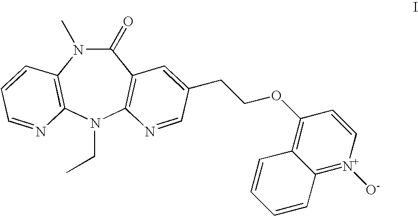

The synthesis and use of the compound of the formula I for the treatment of HIV infection is described in U.S. Pat. No. 6,420,359.

Up until now, there has been little understanding of the metabolism in humans of the compound of the formula I and of the impact which this metabolism may have upon its pharmacokinetics and, by extension, its practical use as a pharmaceutical.

### DESCRIPTION OF THE INVENTION

It has now been discovered that the compound of the formula I is subject to surprisingly rapid metabolism by the cytochromes P450, especially the CYP3A4 isoform. The fact that the compound of the formula I is so rapidly metabolized by cytochromes P450 has, hitherto, been unknown and this fact poses a problem that has, until now, not been appreciated: Metabolism of the compound of the formula I by cytochromes P450 is so rapid as to render it difficult to maintain therapeutically effective blood levels of the compound of the formula I.

The invention provides a solution to this newly recognized problem: It is has been discovered that the pharmacokinetics of the compound of the formula I may be substantially improved by the co-administration of an inhibitor of the cytochromes P450, especially an inhibitor of CYP3A4. It has been found that, when co-administered with an inhibitor of the cytochromes P450, especially an inhibitor of CYP3A4, therapeutically effective blood levels of the compound of the formula I may readily be achieved. Inhibition of the enzymatic activity of the cytochromes P450, especially inhibition of CYP3A4, serves to reduce the metabolism of the compound of the formula I and to thereby substantially improve the pharmacokinetics of the drug, so that less must be administered to attain therapeutic effect. Higher blood levels are also obtained.

Thus, the invention provides an improved method for using the compound of the formula I in the treatment of HIV-1 infection. In its broadest aspect, this method comprises co-administering, to a human needing treatment for HIV-1 infection, an amount of the compound of the formula I or a pharmaceutically acceptable salt thereof, and an amount of at least one pharmaceutically acceptable inhibitor of the cytochromes P450, especially an inhibitor of CYP3A4, which is sufficient to significantly inhibit the enzymatic activity of the cytochromes P450, especially CYP3A4, and to thereby render the amount of the compound of the formula I administered therapeutically effective. Therapeutic effect is deemed to be attained when there is a reduction in the rate of viral replication.

The present invention also provides a method for increasing human blood levels of the compound of the formula I, which comprises co-administering, to a human needing treatment for HIV-1 infection, an amount of the compound of the formula I or a pharmaceutically acceptable salt thereof, and an amount of at least one pharmaceutically acceptable inhibitor of the cytochromes P450, especially an inhibitor of CYP3A4, which is sufficient to significantly inhibit the enzymatic activity of the cytochromes P450, especially CYP3A4, to thereby inhibit drug metabolism and boost and extend exposure to the compound of the formula I.

Consequently the invention provides the use of a combination as described hereinbefore and hereinafter for the manufacture of a medicament for improving the pharmacokinetics of the compound of the formula I.

In addition the invention provides the use of a combination as described hereinbefore and hereinafter for the manufacture of a medicament for increasing the human blood levels of the compound of the formula I.

Furthermore, the invention provides a combination of a therapeutically effective amount of the compound of the formula I or a pharmaceutically acceptable salt thereof and an amount of an inhibitor of the cytochromes P450, which is effective to improve the pharmacokinetics of the compound of the formula I.

The invention also provides a pharmaceutical composition comprising a combination as described hereinbefore and hereinafter and a pharmaceutically acceptable carrier.

In addition the invention provides a kit of parts comprising a combination as described hereinbefore and hereinafter characterized in that

(a) a first containment contains the compound of the formula I or a pharmaceutically acceptable salt thereof and at least one pharmaceutically acceptable carrier, and

(b) a second containment contains the inhibitor of the cytochromes P450 and at least one pharmaceutically acceptable carrier.

The invention also provides a method for the prophylaxis or treatment of HIV infection in a human comprising co-administering to the human in need of such treatment a combination as described hereinbefore and hereinafter.

Thus, the invention also provides the use of a combination as described hereinbefore and hereinafter for the manufacture of a medicament for the prophylaxis or treatment of HIV infection in a human.

In addition the present invention provides the use of the compound of the formula I or a pharmaceutically acceptable salt thereof in the manufacture of a medicament comprising a combination as described hereinbefore and hereinafter for the prophylaxis or treatment of HIV infection in a human.

The invention also provides the use of an inhibitor of the cytochromes P450 in the manufacture of a medicament comprising a combination as described hereinbefore and hereinafter for the prophylaxis or treatment of HIV infection in a human.

In addition the invention provides the use of the compound of the formula I or a pharmaceutically acceptable salt thereof in the manufacture of a medicament for the prophylaxis or treatment of HIV infection in a human in combination with an inhibitor of the cytochromes P450.

Consequently, the invention also provides the use of an inhibitor of the cytochromes P450 in the manufacture of a medicament for the prophylaxis or treatment of HIV infection in a human in combination with the compound of the formula I or a pharmaceutically acceptable salt thereof.

In the context of the invention, it is preferred to inhibit the enzymatic activity of the cytochromes P450, especially CYP3A4, so that this activity is at least halved. To gain the maximum amount of pharmacokinetic improvement possible, it is, however, more preferred to inhibit substantially all of this enzymatic activity.

As used herein, the term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

As used herein the terms “inhibitor of the cytochromes P450” or “inhibitor of CYP3A4” or “CYP 450 inhibitor” refer to any member of the class of pharmaceuticals and/or natural products which inhibit at least the CYP3A4 isoform of the cytochromes P450. The class includes, but is not limited to, amprenavir, atazanavir, clarithromycin, cyclosporin, diltiazem, erythromycin, itraconazole, indinavir, ketoconazole, mibefradil, nefazodone, nelfinavir, ritonavir, vitamin E, bergamottin, dihydroxybergamottin and grapefruit juice. See GK Dresser et al. Clin Pharmacokinetics 2000 Jan; 38(1): 41-57 for a review of clinically-relevant CYP3A4 inhibitors. In the context of the present invention, the preferred inhibitor of CYP3A4 is ritonavir.

As used herein, the term “treatment” means the administration of the antivirally active compounds according to this invention in combination or alternation according to the present invention to alleviate or eliminate symptoms of the viral infection and/or to reduce viral load in a patient.

As used herein, the term “prevention” or “prophylaxis” means the administration of the antivirally active compounds according to this invention in combination or alternation according to the present invention post-exposure of the individual to the virus but before the appearance of symptoms of the disease, and/or prior to the detection of the virus in the blood. The terms “prevention” and “prophylaxis” encompass the prevention of mother-to-child transmission whereby the mother is treated perinatally Oust prior to the birthing process) and optionally during lactation.

It is possible to practice the invention by administering either a single CYP 450 inhibitor or more than one CYP 450 inhibitor. The invention embraces both alternatives.

As used in the context of the present invention, the term “co-administration” refers to the administration of both the compound of the formula I , or a pharmaceutically acceptable salt, and the CYP 450 inhibitor or inhibitors within the same 24 hour period. These drug agents may be administered by means of separate dosage forms or they may be combined into a single dosage form.

Thus the combination according to this invention may comprise the compound of the formula I or a pharmaceutically acceptable salt thereof and the inhibitor of the cytochromes P450 formulated either as a single composition or as a separate composition.

An example of a separate composition is a kit of parts comprising

(a) a first containment which contains the compound of the formula I or a pharmaceutically acceptable salt thereof and at least one pharmaceutically acceptable carrier, and

(b) a second containment which contains the inhibitor of the cytochromes P450 and at least one pharmaceutically acceptable carrier.

In the context of the combinations, methods and uses according to this invention the preferred amount of the compound of the formula I or of a pharmaceutically acceptable salt thereof is a therapeutically effective amount, whereby “therapeutically effective” is to be understood in the context of this invention, i.e. when the compound of the formula I is co-administered with the inhibitor of the cytochromes P450. The preferred amount of the compound of the formula I or of a pharmaceutically acceptable salt thereof is in the range from 50 mg to 3000 mg, in particular in the range from 50 mg to 500 mg, most preferably in the range from 50 mg to 300 mg. In particular a range from 100 mg to 300 is most preferred.

In the context of the combinations, methods and uses according to this invention the preferred amount of the inhibitor of the cytochromes P450 is such that the pharmacokinetics of the compound of the formula I is improved. In the context of this invention the pharmacokinetics of the compound of the formula I is improved when the plasma concentration of said compound of the formula I is elevated, enhanced, or extended compared with an administration of said compound of the formula I not in combination with an inhibitor of the cytochromes P450. Alternatively, it can be said in the context of this invention an improvement of the pharmacokinetics of the compound of the formula I is obtained when the metabolism of the compound of the formula I by the cytochromes P450 is reduced, preferably reduced by at least one third, more preferably reduced by at least one half, most preferably by at least two thirds, compared to the metabolism of the compound of the formula I administered not in combination with an inhibitor of the cytochromes P450.

Furthermore a preferred amount of the inhibitor of the cytochromes P450 is such that the enzymatic activity of the cytochromes P450, especially of the isoform CYP3A4, is reduced, preferably at least halved, in order to improve the pharmacokinetics of the compound of the formula I. Most preferably an amount is chosen such as to inhibit substantially all of this enzymatic activity to gain the maximum amount of pharmacokinetic improvement possible.

In the case wherein ritonavir or a pharmaceutically acceptable salt thereof is chosen as the inhibitor of the cytochromes P450, the preferred amount of the compound of the formula I or its salt is in the range from 30 mg to 1000 mg, in particular in the range from 30 mg to 500 mg, most preferably in the range from 30 mg to 300 mg. In particular a range from 30 mg to 200 mg is most preferred.

Procedures by which the compound of the formula I may be prepared, pharmaceutical compositions comprising the compound of the formula I and its use in the treatment of HIV-1 infection are described in U.S. Pat. No. 6,420,359.

As is described below in Examples 1 and 2, the compound of the formula I coadministered with a sub-therapeutic dose of ritonavir increases the amount of exposure and the length of exposure of the compound of the formula I plasma levels. Coadministration of ritonavir and the compound of the formula I, although resulting in a low blood level of ritonavir, results in the elevation of the compound of the formula I plasma concentration to such an extent that a low dose of the compound of the formula I has a greater therapeutic effect as a much higher dose of the compound of the formula I alone. This is a result of not only boosting the plasma concentration of the compound of the formula I but also retarding the elimination of the compound of the formula I.

Procedures by which ritonavir ((2S,3S,5S)-5-(N-(N-((N-Methyl-N-((2-isopropyl-4-thiazoly)methyl)amino)carbonyl)-L-valinyl)amino)-2-(N-((5-thiazoly)methoxycarbonyl) amino)-1,6-diphenyl-3-hydroxyhexane) may be prepared are described in PCT Patent Application No. WO94/14436, published Jul. 7, 1994, and U.S. patent application Ser. No. 08/469,965, filed Jun. 6, 1995.

The compound of the formula I and inhibitor of the cytochromes P450 used in the methods of the present invention may be in either free form or in protected form at one or more of the remaining (not previously protected) carboxyl, amino, hydroxy, or other reactive groups. The protecting groups may be any of those known in the art. Examples of nitrogen and oxygen protecting groups are set forth in T. W. Greene, Protecting Groups in Organic Synthesis, Wiley, N.Y., (1981); J. F. W. McOmie, ed. Protective Groups in Organic Chemistry, Plenum Press (1973); and J. Fuhrhop and G. Benzlin, Organic Synthesis, Verlag Chemie (1983). Included among the nitrogen protective groups are t-butoxycarbonyl (BOC), benzyloxycarbonyl, acetyl, allyl, phthalyl, benzyl, benzoyl, trityl and the like.

The methods of the present invention provide for the use of pharmacologically acceptable salts and/or hydrates of the compound of the formula I and the inhibitor of the cytochromes P450. Pharmacologically acceptable salts refers to those salts which would be readily apparent to a manufacturing pharmaceutical chemist to be equivalent to the parent compound in properties such as formulation, stability, patient acceptance and bioavailability. Salts of the inhibitor of the cytochromes P450 and the compound of the formula I may include the bis-salts, such as the bis-sodium, bis-potassium and bis-calcium salts, with the bis-sodium salt being most preferred.

The methods of the present invention are useful for treating patients infected with strain 1 of human immunodeficiency virus (HIV-1) which results in acquired immunodeficiency syndrome (AIDS) and related diseases. For this indication, the compound of the formula I and ritonavir may be administered by oral, intranasal, transdermal, subcutaneous and parenteral (including intramuscular and intravenous) routes in doses as described below.

Although a broad range of inhibitors of the cytochromes P450 may be used in the practice of the present invention, ritonavir is, as noted above, the preferred inhibitor. Thus, the invention will now be further illustrated by describing experiments which show, in greater detail, how it may be practiced by the co-administration of the compound of the formula I and ritonavir.

A sub-therapeutic dose of ritonavir of 100 mg, administered 12 hours preceding and co-administered with the compound of the formula I , were investigated in clinical drug-drug interaction studies of ritonavir and the compound of the formula I. The dose of ritonavir studied was shown to have substantial and significant effects on the compound of the formula I by elevating, or enhancing, and extending plasma concentrations of the compound of the formula I. Additionally, plasma concentrations of the compound of the formula I could also be altered by altering the dose of the compound of the formula I , but the extention of plasma concentrations could not be achieved by altering the dose of the compound of the formula I. These results indicate that a target plasma the compound of the formula I can be achieved and maintained through various but well-defined dose combinations of ritonavir. This pharmacokinetic drug interaction is potentially of great clinical importance for a number of reasons, which include:

greater antiviral activity of the compound of the formula I , since antiviral activity is dependent on the magnitude and duration of plasma drug levels,

possibility of reducing the administered the compound of the formula I dose, which may enhance patient compliance to antiviral therapy,

possibly improved safety profile since less the compound of the formula I may be needed to elicit the desired antiviral effect.

The lowest dose of ritonavir tested, 100 mg administered twice daily, was selected on the basis that this is the only available tablet strength of ritonavir commercially available. At this dose level, ritonavir increased plasma concentrations of the compound of the formula I nearly 40-fold as measured by area under the curve.

The half-life of the compound of the formula I without ritonavir was approximately 2 hours over the single dose range of 1-100 mg making clinical use of this entity sub-optimal. Upon co-administration with ritonavir 100 mg, the half-life was extended to 15 hours making the compound of the formula I and low dose ritonavir an attractive drug combination for AIDS therapy.

Those skilled in the art would know how to formulate the compounds of this invention into appropriate pharmaceutical dosage forms. Examples of the dosage forms include oral formulations, such as tablets or capsules, or parenteral formulations, such as sterile solutions.

Either solid or fluid dosage forms can be prepared for oral administration. Solid compositions are prepared by mixing the compounds of this invention with conventional ingredients such as talc, magnesium stearate, dicalcium phosphate, magnesium aluminum silicate, calcium sulfate, starch, lactose, acacia, methyl cellulose, or functionally similar pharmaceutical diluents and carriers. Capsules are prepared by mixing the compounds of this invention with an inert pharmaceutical diluent and placing the mixture into an appropriately sized hard gelatin capsule. Soft gelatin capsules are prepared by machine encapsulation of a slurry of the compounds of this invention with an acceptable inert oil such as vegetable oil or light liquid petrolatum. Syrups are prepared by dissolving the compounds of this invention in an aqueous vehicle and adding sugar, aromatic flavoring agents and preservatives. Elixirs are prepared using a hydroalcoholic vehicle such as ethanol, suitable sweeteners such as sugar or saccharin and an aromatic flavoring agent.

Suspensions are prepared with an aqueous vehicle and a suspending agent such as acacia, tragacanth, or methyl cellulose.

The increase in bioavailability coupled with the extension of half-life has the potential of effectively reducing, by a factor of 30-fold the number of dosing units of the compound of the formula I that are required.

When the compounds of this invention are administered parenterally, they can be given by injection or by intravenous infusion. Parenteral solutions are prepared by dissolving the compounds of this invention in aqueous vehicle and filter sterilizing the solution before placing in a suitable sealable vial or ampule. Parenteral suspensions are prepared in substantially the same way except a sterile suspension vehicle is used and the compounds of this invention are sterilized with ethylene oxide or suitable gas before it is suspended in the vehicle.

The exact route of administration, dose, or frequency of administration would be readily determined by those skilled in the art and is dependant on the age, weight, general physical condition, or other clinical symptoms specific to the patient to be treated.

Having generally described the invention, the same will be more readily understood by reference to the following examples, which are provided by way of illustration and are not intended as limiting.

### EXAMPLE

Pharmacokinetic Drug-Drug Interaction of the compound of the formula I and Ritonavir

Materials and Methods:

A single-dose, single treatment group was studied to assess the pharmacokinetic drug-drug interaction potential between the protease inhibitors the compound of the formula I and ritonavir. The compound of the formula I was administered as a solution containing 5 or 12.5 mg of the compound of the formula I, with excipients, and ritonavir was administered as the 100-mg marketed product (Norvir) 12 hours preceding and co-administration with the compound of the formula I . Baseline pharmacokinetic data for the compound of the formula I was obtained as single doses up through 100 mg. The co-administered drugs were compared with baseline data. The study was conducted in healthy volunteers Pharmacokinetic analyses were based on the results obtained in these subjects.

Pharmacokinetic and Statistical Methods:

Pharmacokinetic parameters such as AUC, Cmax, tmax, oral clearance, and terminal half-life were determined using standard noncompartmental techniques.

Results:

Effects of Ritonavir on the compound of the formula I:

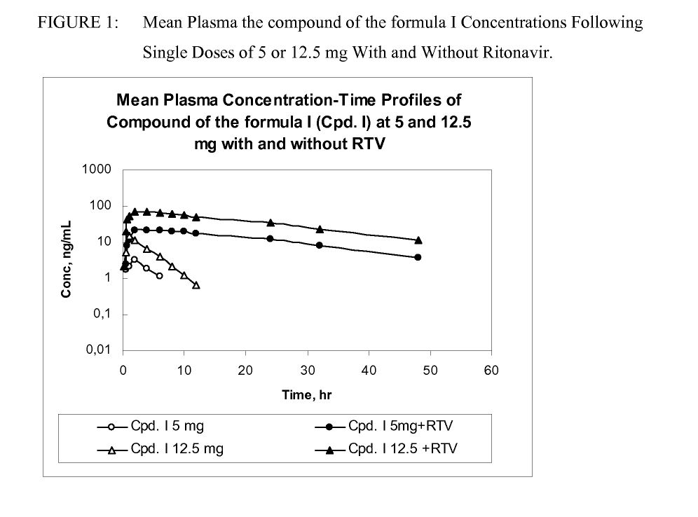

Mean (SD) plasma concentrations of the compound of the formula I following administration of the compound of the formula I alone and in combination with ritonavir (RTV) are shown in TABLE 1 and FIG. 1. The pharmacokinetic estimates of the compound of the formula I up through 100 mg oral dosing is presented in TABLE 2. The mean Cmax value of the compound of the formula I increased approximately 5-6-fold in the presence of ritonavir, whereas mean the compound of the formula I AUC values increased nearly 40-fold because of the prolongation of the half-life from 2 hours to 15 hours (illustrated in FIG. 1).

Discussion:

The results of this study revealed a substantial pharmacokinetic interaction involving both the compound of the formula I and ritonavir. Ritonavir has been shown to both inhibit the metabolism of drugs which are cytochromes P450 3A (CYP3A) substrates (CYP3A is the major P450 isoform for Phase I metabolism of the compound of the formula I ), and to influence absorption through P-glycoprotein inhibition. Likewise, plasma ritonavir concentrations have been shown to be reduced by compounds (such as rifampin) known to induce metabolism.

Lower doses of ritonavir than employed in this study are expected to be sufficient to substantially increase plasma concentrations of the compound of the formula I.

TABLE 1
Effect of Ritonavir on the Pharmacokinetics
of the Compound of the Formula I

| | |
|---|---|
| the compound of the | the compound of the |
| formula I + RTV | formula I alone |

| | | | | |
|---|---|---|---|---|
| PK Parameters | Mean | SD | Mean | SD |

5 mg + RTV

| | | | | |
|---|---|---|---|---|
| AUC 0-t | 577.83 | 121.14 | 13.56 | 10.13 |
| (hr*ng/mL) | | | | |
| Cmax (ng/mL) | 22.79 | 3.70 | 3.64 | 2.23 |
| Tmax (hr) | 4.00 | 1.63 | 1.50 | 0.71 |
| half-life (hr) | 15.30 | 3.25 | —* | — |

12.5 mg + RTV

| | | | | |
|---|---|---|---|---|
| AUC 0-t | 1703.6 | 332.6 | 57.1 | 60.8 |
| (hr*ng/mL) | | | | |
| Cmax (ng/mL) | 74.11 | 14.56 | 14.82 | 14.42 |
| Tmax (hr) | 3.59 | 2.42 | 1.0 | 0 |
| half-life (hr) | 15.89 | 3.32 | 4.10 | 1.91 |

*insufficient data above assay limit to assign a half-life

TABLE 2
Pharmacokinetics of the Compound of the Formula I Alone.

| | | | | | | Half- | | % Renally |
|---|---|---|---|---|---|---|---|---|
| | | | Cmax | Tmax | AUC | life | CL/F | eliminated as |
| Dose | | | (ng/mL) | (hr) | (hr*ng/mL) | (hr) | (mL/min) | unchanged drug |
| 12.5 | mg | mean | 14.82 | 1.00 | 63.15 | 4.10 | 243495.99 | 0.21 |
| | | SD | 14.42 | 0.00 | 68.30 | 1.91 | 118873.29 | 0.12 |
| 25 | mg | mean | 41.63 | 0.83 | 139.36 | 3.44 | 200752.61 | 0.41 |
| | | SD | 29.09 | 0.26 | 71.41 | 1.38 | 80507.44 | 0.32 |
| 50 | mg | mean | 163.81 | 0.67 | 437.37 | 2.50 | 124815.56 | 0.48 |
| | | SD | 39.97 | 0.26 | 122.40 | 0.27 | 39809.58 | 0.30 |
| 75 | mg | mean | 347.68 | 0.67 | 816.70 | 2.21 | 263277.68 | 0.60 |
| | | SD | 222.74 | 0.26 | 550.20 | 0.26 | 400589.38 | 0.43 |
| 100 | mg | mean | 937.23 | 0.55 | 1385.01 | 1.86 | 75634.54 | 0.81 |
| | | SD | 141.66 | 0.21 | 277.01 | 0.24 | 15029.80 | 0.56 |

What is claimed is:

1. A method for treating HIV-1 infection in a human suffering from HIV-1 infection, which method comprises co-administering a compound of the formula I 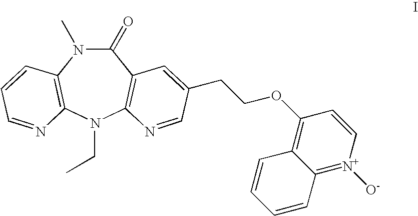
or a pharmaceutically acceptable salt thereof and one or more inhibitors of CYP 450, the latter being administered in an amount which is sufficient to reduce the metabolism of the compound of the formula I by CYP 450 by at least half.

2. The method of claim 1 wherein the amount of the compound of the formula I administered is rendered therapeutically effective by the co-administration of the inhibitor or inhibitors of CYP450.

3 . The method of claim 1 wherein the metabolism by CYP 450 of the compound of the formula I administered is reduced by at least half by the co-administration of the inhibitor of CYP 450.

4. The method of claim 1, 2 or 3 wherein the inhibitor of CYP 450 is selected from the group consisting of amprenavir, atazanavir, clarithromycin, cyclosporin, diltiazem, erythromycin, itraconazole, indinavir, ketoconazole, mibefradil, nefazodone, nelfinavir, ritonavir, vitamin E, bergamottin, dihydroxybergamottin and grapefruit juice.

5. The method of claim 1, 2 or 3 wherein the inhibitor of CYP 450 is ritonavir.

6. A method for improving the pharmacokinetics of the compound of the formula I 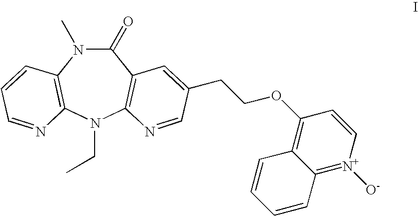
or a pharmaceutically acceptable salt thereof, comprising administering to a human in need of such treatment a combination of the compound of formula I or a pharmaceutically acceptable salt thereof, and ritonavir or a pharmaceutically acceptable salt thereof.

7. The method of claim 6, wherein the amount of the compound of formula I is between about 50 mg and about 3000 mg, and the amount of ritonavir is between about 30 mg and about 1000 mg.

8. The method of claim 6, wherein the amount of the compound of formula I is between about 50 and about 500 mg, and the amount of ritonavir is between about 30 and about 500 mg.

9. The method of claim 6, wherein the amount of the compound of formula I is between about 50 and about 300 mg, and the amount of ritonavir is between about 30 and about 300 mg.

10. The method of claim 6, wherein the amount of the compound of formula I is between about 100 and about 300 mg, and the amount of ritonavir is between about 30 and about 200 mg.

11. A method for increasing human blood levels of the compound of the formula I 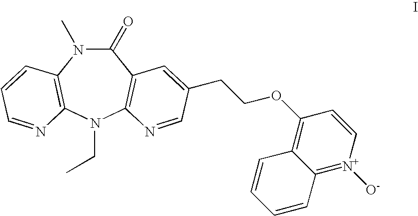
or a pharmaceutically acceptable salt thereof, comprising administering to a human in need of such treatment a combination the compound of the formula I or a pharmaceutically acceptable salt thereof, and ritonavir or a pharmaceutically acceptable salt thereof.

12. The method of claim 11, wherein the amount of the compound of the formula I is between about 200 mg and about 6750 mg, and the amount of ritonavir is between about 30 mg and about 1000 mg.

13. The method of claim 11, wherein the amount of the compound of the formula I is between about 50 and about 3000 mg, and the amount of ritonavir is between about 30 and about 1000 mg.

14. The method of claim 11, wherein the amount of the compound of the formula I is between about 50 and about 500 mg, and the amount of ritonavir is between about 30 and about 500 mg.

15. The method of claim 11, wherein the amount of the compound of the formula I is between about 100 and about 300 mg, and the amount of ritonavir is between about 30 and about 200 mg.
